(19) United States
(12) Patent Application Publication
(10) Pub. No.: US 2026/0218124 A1
(43) Pub. Date: Jul. 30, 2026

(54) A METHOD FOR GENERATION OF PORCINE EMBRYONIC STEM CELLS

(71) Applicant: Myo Palate Corporation, Toronto (CA)

(72) Inventor: Derek Douglas Toms, Guelph (CA)

(21) Appl. No.: 19/139,696

(22) PCT Filed: Dec. 18, 2023
(86) PCT No.: PCT/CA2023/051685

§ 371 Date: Jun. 16, 2025

Related U.S. Application Data
(60) Provisional application No. 63/433,454, filed on Dec. 18, 2022.

(51) Int. Cl. C12N 5/0735; C12N 5/00

(52) U.S. Cl. CPC C12N 5/0606; C12N 5/0081; C12N 2500/14; C12N 2502/13; C12N 2506/04

(57) ABSTRACT

The present invention relates to a method for producing porcine embryonic stem cells, involving the steps of (a) chemically activating a matured porcine oocyte, (b) culturing the chemically activated oocyte until blastocysts are formed, (c) removing the trophoblast from the blastocyst to isolate the inner cell mass (ICM), and (d) transferring the ICM to a culture plate for attachment and cell outgrowth. The present invention also relates to porcine embryonic stem cells obtained through such methods.

### RELATED APPLICATIONS

This application claims priority from U.S. Priority Application No. 63/433,454.

### TECHNICAL FIELD OF INVENTION

This invention relates to the production of porcine embryonic stem cells through chemical activation.

### BACKGROUND

The stable and cost-effective production of porcine embryonic stem cells (“pES cells”) is an area of interest in multiple fields. Porcine embryonic stem cells are suitable for research purposes in animal and human research, for production of research or therapeutic antibodies, and for the production of lab-grown tissue.

For example, the study of early developmental processes are difficult or impractical in livestock, despite a recognized comparative biology and physiology shared between pigs and humans. Therefore, in addition to examining applications to livestock, pES cells can be of interest into research of human disorders. Owing to the similarities between human and pig physiology, pig biomedical models are an available method for investigating human disease and disorders.

Alternatively, pES cells can be used in complex research and development areas, such as the investigation and production of antibodies for research or therapeutic use.

pES further provide benefits for the research into animal husbandry, such as animal treatments, or even animal cloning in the context of endangered pig species. Additionally, the production of porcine embryonic stem cells is of interest in the field of food production.

As the global demand for food increases, the consequences of animal agriculture and meat production are significant. The environmental impacts of agriculture are significant, both in respect of the space required to raise animals, as well as the environmental impacts of growing sufficient feed for the animals.

In addition, ethical concerns over animal husbandry result in a meaningful number of individuals abstaining from consuming animal food products, either outright or in restricted forms.

Meat-substitutes have emerged as an area of focus in recent years. This includes both vegetarian alternatives that are intended to be specifically chosen as an alternative to meats, as well as vegetarian-based meat ‘substitutes’ which seek to mimic an animal product.

Lab-grown meats act as an alternative to animal husbandry, but do not seek to act as ‘substitutes’ for meats. This enables the production of meats with all of the flavor and texture advantages, but in a more ethically and environmentally conscious option compared to the production of meat through animal husbandry.

However, the large-scale production of lab-grown meat is an area where further investigation is necessary, due to difficulties in obtaining stable pES cells, as well as obtaining lab-grown meat cells in a cost-effective manner.

No matter the desired use of the pES cells, there is a need for improved production of stable and cost effective pES cells.

### SUMMARY OF THE INVENTION

The present invention relates to a method for producing porcine embryonic stem cells, involving the steps of (a) chemically activating a matured porcine oocyte, (b) culturing the chemically activated oocyte until blastocysts are formed, (c) removing the trophoblast from the blastocyst to isolate the inner cell mass (ICM), and (d) transferring the ICM to a culture plate for attachment and cell outgrowth.

In one embodiment, chemically activating the matured porcine oocyte is carried out through incubation of the matured porcine oocyte in the presence of a calcium ionophore.

In a further embodiment, chemically activating the matured porcine oocyte is carried out through incubation of the matured porcine oocyte in the presence of a calcium ionophore at concentrations of about 0.5-20 μM.

In a further embodiment, chemically activating a matured porcine oocyte is carried out in the presence of cytochalasin B and/or 6-Dimethylaminopurine.

In a further embodiment, removing the trophoblast from the blastocyst is carried out through the use of immunosurgery.

In a further embodiment, removing the trophoblast from the blastocyst through the use of immunosurgery is carried out through the use of anti-pig serum derived from a rabbit.

In a further embodiment, removing the trophoblast from the blastocyst through the use of immunosurgery is carried out through the use of complement derived from a guinea pig.

In a further embodiment immunosurgery is carried out for about 20-40 minutes at a temperature of about 35-40° C.

In a further embodiment, the culture plate comprises mouse embryonic fibroblast cells.

In an alternative embodiment, the present invention relates to porcine embryonic stem cells obtained via a production method comprising (a) chemically activating a matured porcine oocyte, (b) culturing the chemically activated oocyte until blastocysts are formed, (c) removing the trophoblast from the blastocyst to isolate the inner cell mass (ICM), and (d) transferring the ICM to a culture plate for attachment and cell outgrowth.

In a further embodiment, porcine embryonic stem cells are obtained via a production method comprising chemically activating the matured porcine oocyte through incubation of the matured porcine oocyte in the presence of a calcium ionophore.

In a further embodiment, porcine embryonic stem cells are obtained via a production method comprising chemically activating the matured porcine oocyte through incubation of the matured porcine oocyte in the presence of a calcium ionophore at concentrations of about 0.5-20 μM.

In a further embodiment, porcine embryonic stem cells are obtained via a production method comprising incubating of the matured porcine oocyte in the presence of cytochalasin B and/or 6-Dimethylaminopurine.

In a further embodiment, porcine embryonic stem cells are obtained via a production method comprising removing the trophoblast from the blastocyst through the use of immunosurgery.

In a further embodiment, porcine embryonic stem cells are obtained via a production method comprising removing the trophoblast from the blastocyst through the use of immunosurgery comprising anti-pig serum derived from a rabbit.

In a further embodiment, porcine embryonic stem cells are obtained via a production method comprising removing the trophoblast from the blastocyst through the use of immunosurgery comprising complement derived from a guinea pig.

In a further embodiment, porcine embryonic stem cells are obtained via a production method comprising an immunosurgery step carried out for about 20-40 minutes at a temperature of about 35-40° C.

In a further embodiment, porcine embryonic stem cells are obtained via a production method comprising a culture plate comprises mouse embryonic fibroblast cells.

### BRIEF DESCRIPTION OF DRAWINGS

Steps and elements of the below-described invention are shown in the following figures.

FIG. 1 sets out an image of a hatched blastocyst (upper left) after exiting the zona pellucida (lower right) with the inner cell mass (ICM) visible within the blastocyst.

FIG. 2 shows pig embryos after zona pellucida removal by acidic Tyrode's buffer.

FIG. 3 shows the process of immunosurgery for a single embryo (identified by an arrow in panel i). This involves: ii) zona pellucida removal; iii) antibody binding; iv) complement destruction of the trophoblast; and v) the isolated ICM.

FIG. 4 shows blastocysts before (left) and after (right) trituration with a narrow-bore glass pipette to remove dead cells, debris and remaining zona pellucida from isolated ICM (arrow).

FIG. 5 shows initial ICM attachment. Putative stem cell is indicated with a star.

FIG. 6 shows initial outgrowth from seeded ICM. Putative pES cells are identifiable by their large nucleus and prominent nucleoli.

FIG. 7 shows proliferation of the initial outgrowth (such as those of FIG. 6), following several additional days of culture.

FIG. 8 shows the manual removal of feeder cells after approximately 1 week. ES colonies are preferably divided with a glass needle, and clumps are lifted from the plate.

FIG. 9 shows a clump of unattached pES cells on a fresh plate of feeder cells.

FIG. 10 shows rapid pES growth approximately three days after introduction to a feeder plate.

FIG. 11 shows ES cell colonies expressing active alkaline-phosphatase enzyme, as evidenced by the red colour formed when incubated with fast violet substrate (visible by eye within the well plate).

FIG. 12 shows an ES cell colony (left) stained for the pluripotent marker OCT4 (right).

FIG. 13 shows ES cell colonies stained for the pluripotent markers NANOG (left) and SOX2 (right).

FIG. 14 shows ES cell aggregates differentiated into motor neurons, displaying morphology consistent with dorsal root ganglia.

FIG. 15 shows ES cells differentiated into muscle cells displayed multi-nucleated myotubes (left panel) that stained positive for muscle-specific transcription factor myogenin (center panel) and myosin heavy chain protein (right panel).

### DETAILED DESCRIPTION OF THE INVENTION

Given their unlimited capacity for self-renewal, pig embryonic stem (pES) cells are highly desired for the production of cultured pork as an input cell source as compared to adult stem cells isolated from muscle biopsies. pES cell lines can be established from abattoir-derived ovaries and boar semen. The in vitro generation of blastocyst-stage embryos and chemical modifiers of intracellular signaling pathways that are capable of sustaining the porcine pluripotency network have been previously described (see for example “Chemically Defined Media Can Maintain Pig Pluripotency Network In Vitro” Choi K H, Lee D K, Kim S W, et al. Stem Cell Reports, 2019, hereby incorporated by reference). However, these protocols provide insufficient information to generate pES cells, and fail to provide a suitable source of material.

It has been discovered that pES cells can be prepared in accordance with this invention. Through use of the invention, pES cells can be generated and differentiated into desirable cells capable for use in downstream research, development, or production methods.

### In Vitro Production of Porcine Embryos

Porcine embryos for use in the preparation of pES cells according to the invention can be created through in vitro production methods, such as described in “Culture of pig embryos” (Petters R M and Wells K D. J. Reprod. Fertil. Suppl., 1993); “Birth of Piglets Derived from Porcine Zygotes Cultured in a Chemically Defined Medium” (Yoshioka K, Suzuki C, Tanaka A, et al. Biol. Reprod., 2002); and “Removal of hypotaurine from porcine embryo culture medium does not impair development of in vitro-fertilized or somatic cell nuclear transfer-derived embryos at low oxygen tension” (Chen P R, Spate L D, Leffeler E C, et al. Mol. Reprod. Dev., 2020), each of which is incorporated by reference.

In vitro production of embryos is beneficial in that there is no requirement for in vivo-derived embryos.

Pig ovaries are commonly available from commercial sources, such as a by-product of pork processing in a slaughterhouse. In one embodiment, pig ovaries were obtained, and embryos with antral follicles larger than 4 mm in diameter were punctured with a 18G needle and 10 ml syringe to remove the oocyte-cumulus complexes (COC) and cellular debris.

The cellular debris was exposed to two cycles of saline washes, from which the COCs were isolated under a dissecting microscope and placed into maturation media supplemented with hormones and growth factors (see for example Table 2). Matured oocytes, exemplified by the presence of a polar body, were obtained after culturing in 5% CO₂ in humidified air for approximately 40-50 hours, preferably 42-48 hours, and more preferably 44-48 hours. In one embodiment, oocyte maturation is carried out between about 30-40° C., preferably about 35-38° C., and more preferably at about 38° C.

In some embodiments, once matured the oocytes were denuded of cumulus cells using 0.1% hyaluronidase and trituration. In other embodiments, oocytes were denuded of cumulus cells using other enzymes (such as trypsin) and/or other mechanical methods (such as vortexing).

Matured oocytes can be further processed through the use of either in vitro fertilization (IVF), intracytoplasmic sperm injection (ICSI), electrical activation or chemical activation. In a preferred embodiment, the matured oocytes are further processed using chemical activation.

### Chemical Activation of Matured Oocytes

It has been discovered that the use of parthenogenesis via chemical activation increases the number of embryos that can be obtained per oocyte compared to other in vitro productions methods (such as IVF or ICSI).

Chemical activation takes place through incubation of matured oocytes in embryo culture (EC) media. One suitable EC media for use in the invention is shown in Table 3.

It has been discovered that chemical activation can be best achieved through the use of a modified NCSU base media when formulating EC media. In one embodiment, chemical activation is carried out by a calcium-binding compound, preferably a cell-permeable calcium-binding compound such as a calcium ionophore. In a particularly preferred embodiment, the calcium ionophore used is “Calcium ionophore A23187”.

Calcium ionophore is present in concentrations of 0.5-20 μM, preferably 1-10 μM, more preferably 5 μM. Preferably, oocytes are incubated for 1-30 minutes in the presence of calcium ionophore, more preferably 5-20 minutes, more preferably 5-10 minutes, and most preferably 7 minutes.

In one embodiment, the EC media further comprises one or more antibiotic or antimicrobial ingredients, preferably penicillin and/or streptomycin.

In another embodiment, chemical activation further comprises the presence of one or more of cytochalasin B and 6-Dimethylaminopurine (DMAP), to prevent second polar body extrusion and allow for the development of diploid parthenogenic blastocysts.

Cytochalasin B concentrations can range from 1-10 μg/ml, preferably 5-9 μg/ml, and more preferably 7.5 μg/ml. DMAP concentrations can range from 0.5-5 mM, preferably 1-3 mM, and more preferably 2 mM.

Preferably, activated oocytes are incubated with CB and/or DMAP for about 2-10 hours, preferably for about 4-8 hours, and more preferably for about 4-5 hours.

In preferred embodiments, EC media is used for the chemical activation step. In a particularly preferred embodiment, the EC media used is as described in Table 3.

In a further embodiment, following 24 h of chemical activation, zygotes were cleaved and transferred to an embryo culture plate and cultured in groups of 30-100 for five additional days at 38.5° C. in 5% CO₂, 5% O₂ in humidified air. Within 8 days of incubation, embryos reached the blastocyst stage.

The above methods have been discovered to provide for a suitable supply of blastocysts for further use in accordance with the invention.

### Derivation of Pig Embryonic Stem Cells

It has further been discovered that pES cells can be obtained from porcine blastocysts, such as blastocysts produced in accordance with the above described embodiments.

Blastocysts are selected after undergoing 5-10 days of development after activation, preferably 5-8 days, and more preferably 6-8 days.

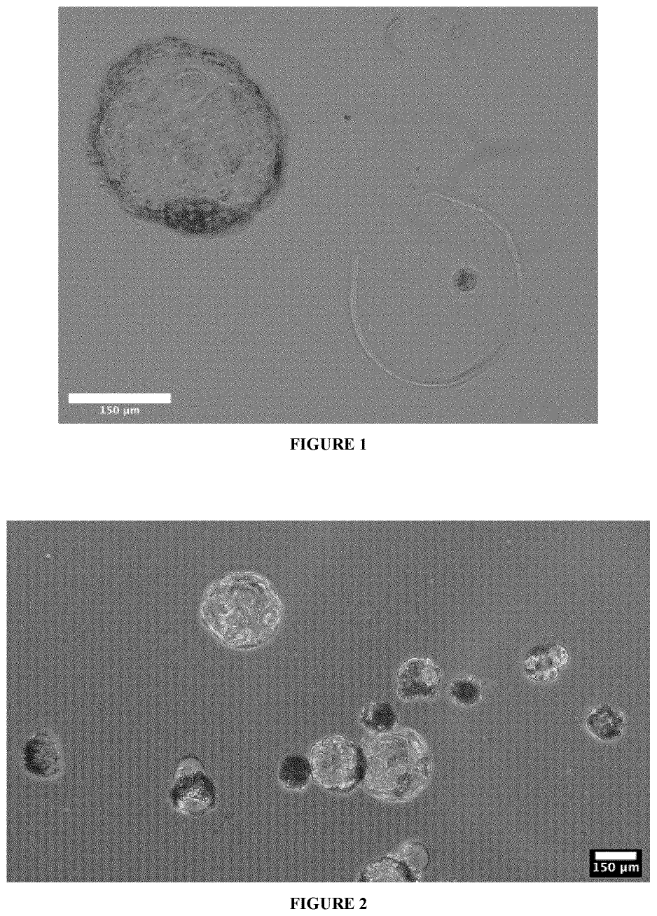

Blastocysts at this stage post-activation should have hatched from the zonal pellucida, as shown in FIG. 1. In one embodiment, a wash with acidic Tyrode lactate (ATL) buffer can be used to remove or sufficiently thin any remaining zona pellucida. Zona pellucida can be thinned or removed by incubating the blastocysts in ATL for about 1 to 5 minutes. In one embodiment, the blastocysts are incubated until the zona pellucida is less than 5 μm thick. In a preferred embodiment, blastocysts are incubated until no zona pellucida is present, as shown in FIG. 2.

In another embodiment, the zona pellucida can also be mechanically or enzymatically removed, such as through the use of glass needles, laser micromachinery, or other known techniques. In a preferred embodiment, mechanical removal is done after the zona pellucida is first thinned as described above.

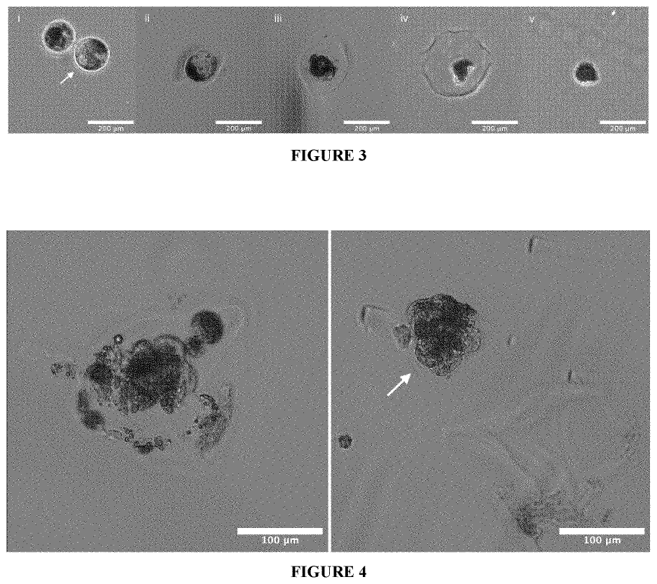

It has been discovered that blastocysts are preferably processed to remove the trophoblast through the use of antibody-mediated complement cell destruction (also known as “immunosurgery”). Immunosurgery allows for isolation of the inner cell mass (ICM) to obtain the pES cells, as shown in FIG. 3. Similar to zona pellucida removals as described above, mechanical dissection or enzymatic digestion of the blastocysts to isolate ICM can be used, either in addition to or as an alternative process. Under any approach, care should be taken to ensure the ICM is not harmed by the process.

Immunosurgery in accordance with the invention can be carried out using any anti-pig serum (or recombinant antibodies), and any complement diluted in suitable media. Preferred sources of anti-pig serum are obtained from rabbits, goats, or sheep. Preferred sources of recombinant antibodies (mono- or poly-clonal) are obtained from rodent or rabbit immortalized cells. Preferred sources of complement are obtained from guinea pigs, mice, rats, rabbits, moneys, and humans. In a particularly preferred embodiment, immunosurgery is carried out using rabbit serum and guinea pig complement.

In a preferred embodiment, immunosurgery is carried out by incubating blastocysts in anti-pig rabbit serum (most preferably a 1:10 dilution of serum with wash buffer). Incubation preferably takes place for 20-40 minutes, preferably for 30 minutes. Incubation preferably takes place between about 35-40° C., more preferably at 37° C.

In a preferred embodiment, blastocysts are placed in a 1:10 dilution of complement in Embryo wash buffer (such as that of Table 5) and further incubated for 20-40 minutes, more preferably 30 minutes, at an incubation temperature between about 35-40° C., more preferably about 37° C. The species of the complement used can be the same as or different from the species of the serum. In a preferred embodiment a different species is used. In a particularly preferred embodiment guinea pig complement is used.

In a further embodiment the immunosurgery step includes a wash step between the steps of adding serum (or recombinant antibodies) and adding complement, to remove any residual and unbound antibody. In a preferred embodiment, this wash step is done more than once, for up to about 5 minutes per wash. In a preferred embodiment, this wash step is done three times at about 5 minutes per wash.

Following immunosurgery and optional wash steps the ICM must be cleaned for better attachment to, and outgrowth from, a culture plate. ICM cleaning is preferably performed by triturating the ICM with a narrow-bore glass pipette up to 50 times to remove residual zona pellucida fragments, dead/dying trophoblast cells, as shown in FIG. 4. Other means for removal, such as the use of glass needles or laser dissection, can alternatively or additionally be used.

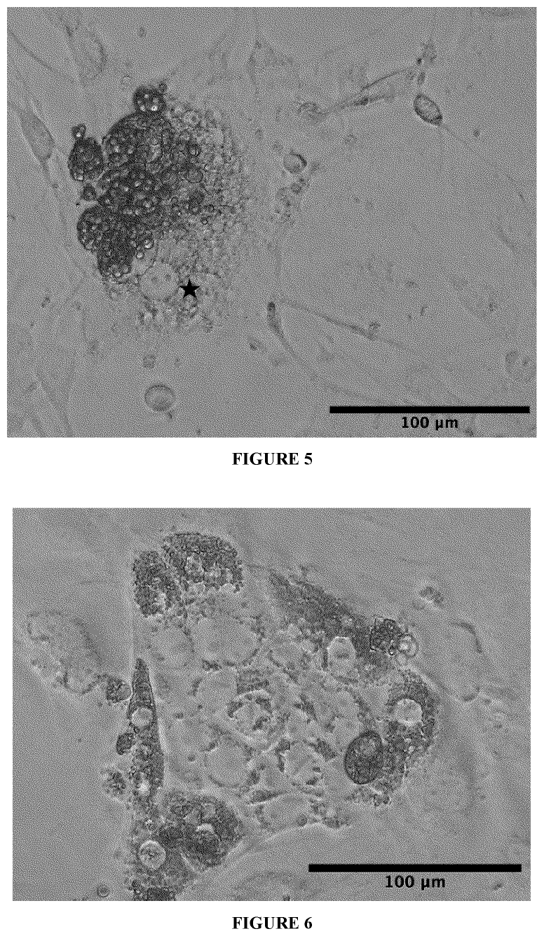

When ICM cleaning is completed, individual ICMs are placed onto culture plates to allow for attachment and cell outgrowth, which represents the pES cell, seen in FIG. 5. In one embodiment, the culture plates comprise extracellular proteins such as fibronectin or laminin. In a preferred embodiment, the culture plates comprise a feeder culture of fibroblast cells, such as mouse, rat, rabbit, or pig fibroblast cells. In a particularly preferred embodiment, the culture plates comprise a feeder culture of mouse embryonic fibroblast cells.

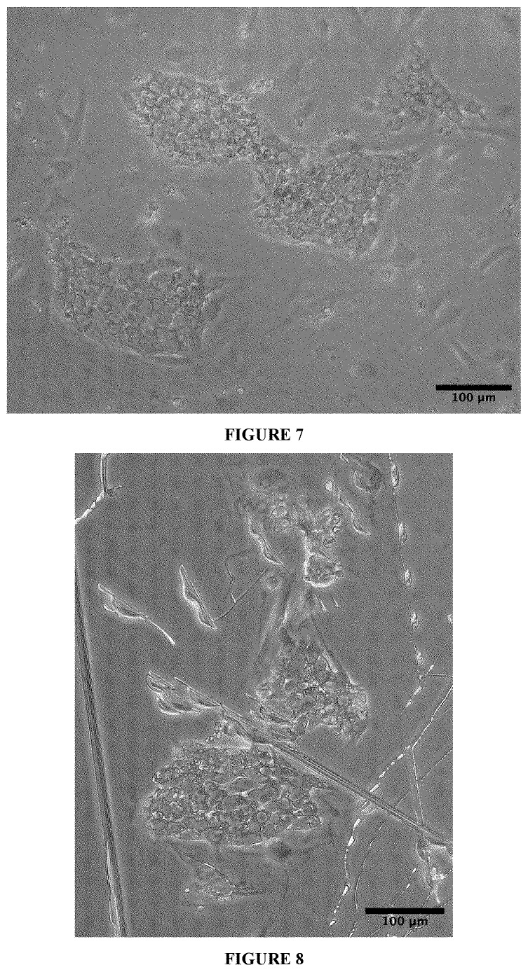

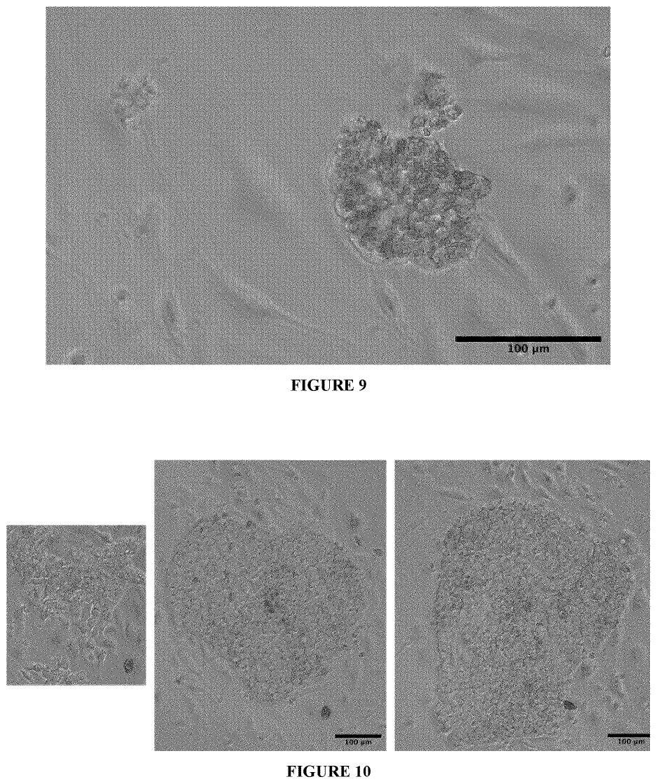

After the ICM has attached, outgrowths (i.e., small clumps of cells) must be observed, such as those shown in FIGS. 5-7. Outgrowths of cells are then lifted from the culture plate, through chemical, enzymatic, or mechanical means, and re-seeded onto fresh culture plates. In a preferred embodiment, the outgrowths are lifted through mechanical means and seeded onto mouse fibroblast feeder cells, as shown in FIGS. 8 and 9.

In a preferred embodiment, the media for ICM attachment consists of Embryo Derivation Media (such as that of Table 6).

In another preferred embodiment, no media change is performed during the first seven days of attachment to provide for maximal attachment and outgrowth, as shown in FIG. 10.

### Confirmation of pES Cell Differentiation

When processed in accordance with the invention as described above, it has surprisingly been discovered that a stable and differentiated pES can be obtained. Confirmation of pES differentiation can be performed through a variety of techniques.

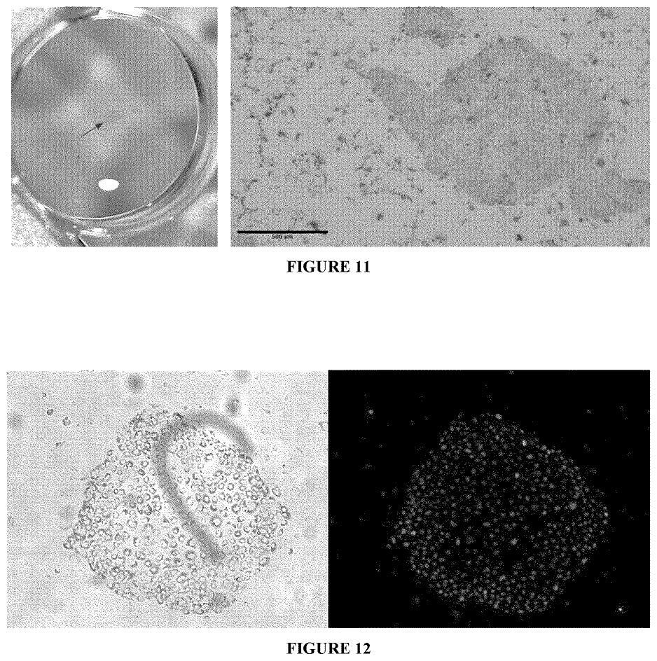

In one embodiment, pES differentiation is observed visually, through the observance of a red colour when cells are incubated with fast violet substrate, as shown in FIG. 11. The red colour is obtained due to the presence of expressed active alkaline-phosphatase enzyme.

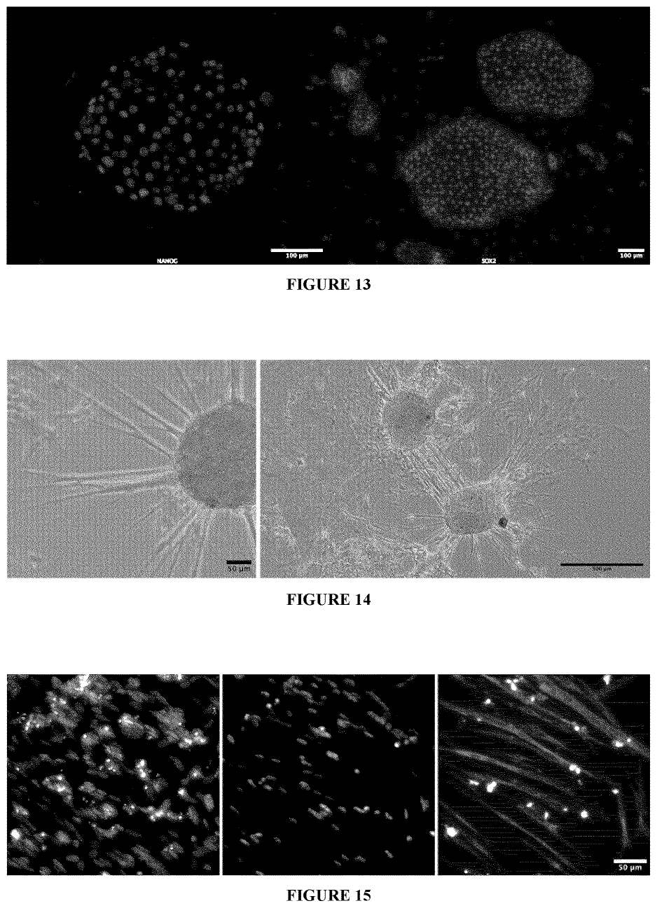

In another embodiment, pES cells can be stained for one or more of the pluripotent markers OCT4, NANOG, or SOX2, seen in FIGS. 12 and 13.

In a further embodiment, pES cells will show differentiated motor neurons displaying morphology consistent with dorsal root ganglia, when observed under a microscope, as shown in FIG. 14.

In yet another embodiment, pES cells can be stained for the presence of myosin heavy chain protein, and/or muscle-specific transcription factor myogenin, seen in FIG. 15.

### List of Materials and Reagents for Use with the Disclosed Invention

The following materials, solutions, and media are suitable for use in the preparation of materials in accordance with the invention. Suitable alternatives to the listed reagents are also known, and such changes do not detract from the invention.

TABLE 1
In vitro Maturation (IVM) Media:

| | Concentration | | Preferred | |
|---|---|---|---|---|
| Reagent Description | Range* | | Concentration | |
| Medium 199, Earle's Salts | solvent | | — | |
| D-glucose | 1-5 | mM | 3.05 | mM |
| sodium pyruvate | 0.1-2 | mM | 0.91 | mM |
| polyvinyl alcohol (PVA) | 0.01-0.5 | (w/v) | 0.1% | (w/v) |
| L-cysteine | 0.2-2 | mM | 0.57 | mM |
| follicle-stimulating hormone | 0-0.1 | U/mL | 0.01 | U/mL |
| (FSH) | | | | |
| luteinizing hormone (LH) | 0-0.1 | U/mL | 0.01 | U/mL |
| recombinant EGF | 1-50 | ng/mL | 10 | ng/mL |
| recombinant FGF2 | 10-100 | ng/mL | 40 | ng/mL |
| recombinant LIF | 1-50 | ng/mL | 20 | ng/mL |
| recombinant IGF1 | 1-50 | ng/mL | 20 | ng/mL |

Adjust pH to between 7.2-7.4. Sterile-filter media (0.22 μm) and store at 4° C. for up to four weeks.
*A concentration range encompassing a “0” value denotes an optionally added reagent.

TABLE 2
Modified North Carolina State University (mNCSU) Media:

| | Concentration | | Preferred | |
|---|---|---|---|---|
| Reagent Description | Range* | | Concentration | |
| ultrapure water | solvent | | — | |
| sodium chloride | 80-150 | mM | 108.7 | mM |
| potassium chloride | 0-10 | mM | 4.78 | mM |
| calcium chloride dihydrate | 0-10 | mM | 1.70 | mM |
| potassium phosphate monobasic | 0-10 | mM | 1.19 | mM |
| magnesium sulfate heptahydrate | 0-10 | mM | 1.19 | mM |
| sodium bicarbonate | 0-100 | mM | 25.07 | mM |
| L-glutamine | 0-10 | mM | 1.00 | mM |
| taurine | 0-50 | mM | 12.00 | mM |
| sodium pyruvate | 0-1 | mM | 0.182 | mM |
| sodium DL-lactate solution | 0-10 | mM | 2.70 | mM |

Adjust pH to between 7.3-7.4 and osmolarity to between 280-290 mOsm. Sterile-filtered mNCSU media (0.22 μm) can be stored at 4° C. for up to four weeks.
*A concentration range encompassing a “0” value denotes an optionally added reagent.

TABLE 3
Embryo Culture (EC) Media:

| Reagent Description | Concentration Range* | | Preferred Concentration | |
|---|---|---|---|---|
| mNCSU | solvent, see Table 2 | | — | |
| bovine serum albumin | 0-1% | (w/v) | 0.4% | (w/v) |
| D-glucose | 0-10 | mM | 5.55 | mM |

Adjust pH to between 7.3-7.4 and osmolarity to between 280-290 mOsm. Sterile-filtered media (0.22 μm) can be stored at 4° C. for up to four weeks.
*A concentration range encompassing a “0” value denotes an optionally added reagent.

TABLE 4
Acidic Tyrode Lactate (ATL) Buffer:

| | | |
|---|---|---|
| | Concentration | Preferred |
| Reagent Description | Range* | Concentration |

| | | |
|---|---|---|
| ultrapure water | solvent | — |

| | | | | |
|---|---|---|---|---|
| sodium chloride | 100-150 | mM | 136.9 | mM |
| potassium chloride | 0-10 | mM | 2.68 | mM |
| calcium chloride | 0-10 | mM | 1.63 | mM |
| magnesium chloride | 0-2 | mM | 0.492 | mM |
| D-glucose | 0-10 | mM | 5.55 | mM |
| polyvinylpyrrolidone | 0-1% | (w/v) | 0.4% | (w/v) |

Adjust pH to between 27.43-27.64 and osmolarity to between 280-290 mOsm. Sterile-filtered media (0.22 μm) can be stored at 4° C. for up to four weeks.
*A concentration range encompassing a “0” value denotes an optionally added reagent.

TABLE 5
Embryo Wash Buffer:

| | | |
|---|---|---|
| | Concentration | Preferred |
| Reagent Description | Range* | Concentration |

| | | |
|---|---|---|
| ultrapure water | solvent | — |

| | | | | |
|---|---|---|---|---|
| sodium chloride | 90-150 | mM | 120 | mM |
| potassium chloride | 0-10 | mM | 3.21 | mM |
| sodium phosphate dibasic | 0-2 | mM | 0.394 | mM |
| sodium DL-lactate solution | 0-10 | mM | 2.70 | mM |
| magnesium chloride | 0-2 | mM | 0.492 | mM |
| sodium bicarbonate | 0-5 | mM | 2.00 | mM |
| HEPES sodium salt | 0-12 | mM | 9.22 | mM |
| polyvinyl alcohol (PVA) | 0-0.5% | (w/v) | 0.1% | (w/v) |

Adjust pH to between 7.3-7.4 and osmolarity to between 280-290 mOsm. Sterile-filtered media (0.22 μm) can be stored at 4° C. for up to four weeks.
*A concentration range encompassing a “0” value denotes an optionally added reagent.

TABLE 6
Embryo Derivation Media:

| | Concentration | | Preferred | |
|---|---|---|---|---|
| Reagent Description | Range* | | Concentration | |
| KnockOut ™ DMEM | solvent | | — | |
| KnockOut ™ Serum Replacement | 2-25% | (v/v) | 20% | (v/v) |
| GlutaMAX ™ Supplement (L- | 0-5 | mM | 2 | mM |
| alanyl-L-glutamine dipeptide in | | | | |
| 0.85% NaCl) | | | | |
| MEM Non-essential amino acids | 0.05-0.2 | mM | 0.1 | mM |
| solution (mixture of glycine, | | | | |
| alanine, asparagine, aspartic acid, | | | | |
| glutamic acid, proline, serine) | | | | |
| Chemically Defined Lipid | 0-1% | (v/v) | 0.1% | (v/v) |
| Concentrate (mixture of lipids | | | | |
| and surfactants) | | | | |
| 2-Mercaptoethanol | 0-0.5 | mM | 0.1 | mM |

Adjust pH to between 7.3-7.4 and osmolarity to between 280-290 mOsm. Sterile-filtered media (0.22 μm) can be stored at 4° C. for up to four weeks.
*A concentration range encompassing a “0” value denotes an optionally added reagent.

### REFERENCES

The following references may be of assistance to the reader and are hereby incorporated by reference in their entirety:
a. J. A. Piedrahita, G. B. Anderson, and R. H. Bondurant, Influence of feeder layer type on the efficiency of isolation of porcine embryo-derived cell lines. Theriogenology (1990).
b. M. Serrano Albal, G. Silvestri, L. G. Kiazim, et al., Supplementation of porcine in vitro maturation medium with FGF2, LIF, and IGF1 enhances cytoplasmic maturation in prepubertal gilts oocytes and improves embryo quality. Zygote (2022).
c. S. Liu, K. Cui, H. L. Li, et al., Comparison of chemical, electrical, and combined activation methods for in vitro matured porcine oocytes. In Vitro Cell Dev Biol Anim (2015).
d. M. A. Silvestre, J. Alfonso, E. García-Mengual, et al., Effect of recombinant human follicle-stimulating hormone and luteinizing hormone on in vitro maturation of porcine oocytes evaluated by the subsequent in vitro development of embryos obtained by in vitro fertilization, intracytoplasmic sperm injection, or parthenogenetic activation. J Anim Sci (2007).
e. Y. Yuan, L. D. Spate, B. K. Redel, et al., Quadrupling efficiency in production of genetically modified pigs through improved oocyte maturation. Proc Natl Acad Sci USA (2017).
f. K.-H. Choi, D.-K. Lee, S. W. Kim, et al., Chemically Defined Media Can Maintain Pig Pluripotency Network In Vitro. Stem Cell Reports (2019).
g. Petters R M and Wells K D. J., Culture of pig embryos, Reprod. Fertil. Suppl., 1993.
h. Yoshioka K, Suzuki C, Tanaka A, et al., Birth of Piglets Derived from Porcine Zygotes Cultured in a Chemically Defined Medium, Biol. Reprod., 2002
i. Chen P R, Spate L D, Leffeler E C, et al., Removal of hypotaurine from porcine embryo culture medium does not impair development of in vitro-fertilized or somatic cell nuclear transfer-derived embryos at low oxygen tension, Mol. Reprod. Dev., 2020

It is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims. It is readily apparent to one skilled in the art that various embodiments and modifications can be made to the disclosure of the present application without departing from the invention.

1.-10. (canceled)

11. A method for producing porcine embryonic stem cells, said method comprising:
(a) chemically activating a matured porcine oocyte;
(b) culturing the chemically activated oocyte until blastocysts comprising trophoblasts are formed;
(c) removing the trophoblast from the blastocyst to isolate the inner cell mass (ICM) of the blastocyst; and
(d) transferring the ICM to a culture plate for attachment and cell outgrowth.

12. The method of claim 11, wherein chemically activating the matured porcine oocyte is carried out through incubation of the matured porcine oocyte in the presence of calcium ionophore.

13. The method of claim 12, wherein calcium ionophore is present at a concentration of about 0.5-20 μM.

14. The method of claim 11, wherein chemically activating a matured porcine oocyte is carried out in the presence of cytochalasin B and/or 6-Dimethylaminopurine.

15. The method of claim 14, wherein cytochalasin B is present at a concentration of about 5-9 μg/mL.

16. The method of claim 14, wherein 6-Dimethylaminopurine is present at a concentration of about 1-3 mM.

17. The method of claim 11, wherein removing the trophoblast from the blastocyst is carried out through the use of mechanical dissection.

18. The method of claim 11, wherein removing the trophoblast from the blastocyst is carried out through the use of immunosurgery.

19. The method of claim 18, wherein the immunosurgery comprises the use of anti-pig serum derived from a rabbit.

20. The method of claim 18, wherein the immunosurgery comprises the use of complement derived from a guinea pig.

21. The method of claim 18, wherein immunosurgery is carried out for about 20-40 minutes at a temperature of about 35-40° C.

22. The method of claim 11, wherein the culture plate comprises mouse embryonic fibroblast cells.

23. The method of claim 11, wherein the culture plate comprises a coating of extracellular proteins.

24. The method of claim 11, wherein:
(a) chemically activating the matured porcine oocyte is carried out through incubation of the matured porcine oocyte in the presence of:
(ii) calcium ionophore at a concentration of about 0.5-20 μM; and
(ii) cytochalasin B and/or 6-Dimethylaminopurine;
(b) removing the trophoblast from the blastocyst is carried out through the use of immunosurgery comprising anti-pig serum derived from a rabbit or complement derived from a guinea pig, for about 20-40 minutes at a temperature of about 35-40° C.; and
(c) the culture plate comprises mouse embryonic fibroblast cells.

25. The method of claim 11, wherein:
(a) chemically activating the matured porcine oocyte is carried out through incubation of the matured porcine oocyte in the presence of:
(i) calcium ionophore at a concentration of about 0.5-20 μM; and
(ii) cytochalasin B and/or 6-Dimethylaminopurine;
(b) removing the trophoblast from the blastocyst is carried out through the use of mechanical dissection; and
(c) the culture plate comprises mouse embryonic fibroblast cells.

26. The method of claim 11, wherein:
(a) chemically activating the matured porcine oocyte is carried out through incubation of the matured porcine oocyte in the presence of:
(i) calcium ionophore at a concentration of about 0.5-20 μM; and
(ii) cytochalasin B and/or 6-Dimethylaminopurine;
(b) removing the trophoblast from the blastocyst is carried out through the use of immunosurgery comprising anti-pig serum derived from a rabbit or complement derived from a guinea pig, for about 20-40 minutes at a temperature of about 35-40° C.; and
(c) the culture plate comprises a coating of extracellular proteins.

27. The method of claim 11, wherein:
(a) chemically activating the matured porcine oocyte is carried out through incubation of the matured porcine oocyte in the presence of:
(i) calcium ionophore at a concentration of about 0.5-20 μM; and
(ii) cytochalasin B and/or 6-Dimethylaminopurine;
(b) removing the trophoblast from the blastocyst is carried out through the use of mechanical dissection; and
(c) the culture plate comprises a coating of extracellular proteins.

28. The method of claim 11, wherein:
(a) chemically activating the matured porcine oocyte is carried out through incubation of the matured porcine oocyte in the presence of:
(i) calcium ionophore at a concentration of about 0.5-20 μM; and
(ii) cytochalasin B and/or 6-Dimethylaminopurine; and
(b) the culture plate comprises mouse embryonic fibroblast cells.

29. The method of claim 11, wherein:
(a) chemically activating the matured porcine oocyte is carried out through incubation of the matured porcine oocyte in the presence of:
(i) calcium ionophore at a concentration of about 0.5-20 μM; and
(ii) cytochalasin B and/or 6-Dimethylaminopurine; and
(b) the culture plate comprises a coating of extracellular proteins.
